(19) United States
(12) Patent Application Publication
(10) Pub. No.: US 2005/0163752 A1
(43) Pub. Date: Jul. 28, 2005

(54) Human interferon-beta formulations

(76) Inventors: Michael McCaman, Fairfield, CA (US); Susanne Ottoboni, Belmont, CA (US); Erno Pungor, Millbrae, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/063,597

(22) Filed: Feb. 24, 2005

Related U.S. Application Data
(63) Continuation of application No. 10/190,838, filed on Jul. 9, 2002, now abandoned.
(60) Provisional application No. 60/303,395, filed on Jul. 9, 2001.

(51) Int. Cl. A61K 38/21; A61K 31/198

(52) U.S. Cl. 424/85.6; 514/561

(57) ABSTRACT

The invention provides a stable pharmaceutical composition containing biologically active human interferon-β (IFN-β), preferably IFN β-1b produced in a bacterial host, dissolved in an aqueous-based solution containing a glycine buffer at a pH of about 2.0 to about 4.0. The invention of also provides stable IFN-β lyophilizates prepared from biologically active IFN-β, dissolved in an aqueous-based solution containing a glycine buffer at a pH of about 2.0 to about 4.0.

This invention relates, e.g, to pharmaceutical formulations for interferon-β which comprise a glycine buffer at pH about 2.0 to about 4.0 and which do not contain substantial amounts of human serum albumin or detergent.

### BACKGROUND OF THE INVENTION

Interferon-β (“IFN-β”) is used to treat several medical conditions and is being investigated for a number of others. For the majority of purposes, recombinantly-produced human IFN-β is used. In particular, a genetically engineered version of human IFN-β in which Ser¹⁷ replaces Cys¹⁷ (“IFN β-1b”), as described in U.S. Pat. No. 4,588,585 has been approved for treatment of multiple sclerosis.

### SUMMARY OF THE INVENTION

The present invention relates, e.g., to low pH (e.g., pH about 2.0 to about 4.0) interferon-β (IFN-β) compositions comprising a glycine buffer. The compositions of the invention are stable as liquid formulations and as lyophilizates in the substantial absence of conventional stabilizers (e.g., human serum albumin) and/or solubilizers (e.g., detergents). The invention particularly relates to biologically active human IFN-β, preferably recombinant IFN-β, including IFN-β analogs, and most preferably IFN β-1b, as described in U.S. Pat. No. 4,588,585.

One aspect of the invention is an IFN-β composition comprising biologically active IFN-β to which a glycine buffer has been added to achieve a pH of about 2 to about 4, e.g., wherein the buffer further comprises HCl; a composition having a pH of about 2 to about 4, comprising biologically active IFN-β and a glycine buffer or biologically active IFN-β and glycine; an IFN-β composition consisting essentially of biologically active IFN-β to which a glycine buffer has been added to achieve a pH of about 2 to about 4, e.g., wherein the buffer comprises HCl; or a composition having a pH of about 2 to about 4, consisting essentially of biologically active IFN-β, water and a glycine buffer, or biologically active IFN-β, water and glycine. The water in the compositions of the invention is preferably sterile water which is, e.g., substantially free of pyrogens or trace minerals, most preferably USP grade water for injection (WFI).

Another aspect of the invention is any of the above IFN-β compositions, wherein the glycine is in a stabilizing effective amount; wherein the composition is in the form of a pharmaceutical composition, is sterile, or is in a container for parenteral or subcutaneous administration (e.g., injection or inhalation); wherein at least 75% of the biological activity of the IFN β-1b is retained after storage of the composition at 4° C. for at least 9 months; wherein the IFN-β is unglycosylated and is produced in a bacterial host, e.g., is IFN β-1b; wherein the composition is substantially free of human serum albumin or detergent and/or is in the substantial absence of glycerol or PEG; wherein the concentration of biologically active IFN-β is between about 1.0 mg/mL and about 20 mg/mL; and/or wherein the IFN-β is not in the form of a non-covalently associated aggregate.

Another aspect of the invention is a lyophilized IFN-β composition consisting essentially of biologically active IFN-β and glycine/HCl or biologically active IFN-β and glycine; or comprising biologically active IFN-β and glycine. The invention also relates to any of the above lyophilized IFN-β compositions, wherein the IFN-β is unglycosylated and is produced in a bacterial host, e.g., is IFN β-1b; or wherein at least 75% of the biological activity of the IFN-β is recoverable in soluble form after storage of the composition at about 25° C. at least 6 months. The invention also relates to a lyophilized IFN-β composition prepared by lyophilizing a solution having a pH of about 2 to about 4, which consists essentially of biologically active IFN-β, water (e.g., WFI) and a glycine buffer, to obtain said lyophilized IFN-β composition; or prepared by lyophilizing a solution having a pH of about 2 to about 4, which comprises biologically active IFN-β, water (e.g., WF) and a glycine buffer, to obtain said lyophilized IFN-β composition.

Another aspect of the invention is a process for preparing a lyophilized IFN-β composition, comprising lyophilizing a solution having a pH of about 2 to about 4, consisting essentially of biologically active IFN-β, water (e.g., WFI) and a glycine buffer, to obtain said lyophilized IFN-β; or comprising lyophilizing a solution having a pH of about 2 to about 4, comprising biologically active IFN-β and a glycine buffer, to obtain said lyophilized IFN-β composition; or to either of the above processes, wherein the IFN-β is unglycosylated and is produced in a bacterial host, e.g., is IFN β-1b.

Another aspect of the invention is a kit comprising a) a container which contains a lyophilized IFN-β composition as above and b) a container which contains a suitable aqueous solution for reconstituting said composition (e.g., sterile water, preferably sterile, pyrogen-free water, most preferably WFI).

In a most preferred embodiment, the composition comprises, or consists essentially of about 5 mg/mL biologically active IFN β-1b in about 0.02M glycine/HCl buffer at pH about 3.0.

Surprisingly, it has been found that a buffered solution with a pH of about 2.0 to about 4.0, preferably about 3.0 to about 4.0, more preferably about 3.0 to about 3.5, and most preferably about 3.0 provides excellent stability and solubility for IFN-β in liquid formulation or as a lyophilizate. In a preferred embodiment, the buffer is a glycine buffer which comprises, in addition to glycine, HCl. However, many other types of buffers can be used (e.g., aspartic acid or glutamic acid); and many other types of acids can be used to adjust the pH (e.g., phosphoric acid). The discussion herein focuses primarily on glycine/HCl buffers. However, one of skill in the art will recognize that this is only exemplary of the many types of buffers which can be used.

An advantage of the buffers of the invention is that they impart stability and/or solubility to IFN-β, even in the substantial absence of conventional stabilizers and/or solubilizers, such as e.g., human serum albumin (HSA); high molecular weight or polyalcohol solubilizers/stabilizers such as polyethylene glycols (PEG), glycerol, polyhydric sugar alcohol, or polyvinylpyrrolidone; or the like, as described, e.g., in U.S. Pat. Nos. 5,643,566, 5,004,605, 3,981,991 or 4,496,537, EP 080 879 or 082 481 A, or BE 897,276. Such stabilizers and solubilizers are disadvantageous in pharmaceutical compositions because they add to the cost of preparation of the compositions, can cause allergic reactions, and may not be compatible with preferred pH conditions for processing, lyophilization and lyophilizate reconstitution. Components of the buffers of the instant invention, e.g. glycine, are present in the compositions in stabilizing-effective amounts.

Solubilizers such as SDS, which are used to solubilize the inclusion bodies in which heterologous proteins such as IFN-β are often produced in an aggregated or denatured form by bacteria, must be removed from the heterologous protein during processing, as such solubilizers are toxic and/or denature the biologically active form of the heterologous protein, e.g., by unfolding the native structure of the heterologous protein. However, heterologous proteins produced by bacteria, and particularly IFN-β produced by bacteria, are subject to solubility problems after removal of the solubilizer or SDS. An advantage of the present invention is that it provides a stable solution of soluble, biologically active recombinant IFN-β even in the substantial absence of detergent and/or solubilizer such as, e.g., SDS or Zwit 314.

Buffers of the invention also minimize the formation of non-covalently associated multimers or aggregates of IFN-β (i.e., they optimize the formation of non-covalently associated monomers). The degree of aggregation can be determined by conventional methods such as, e.g., dynamic light scattering or size exclusion chromatography. Because compositions of the invention are substantially free of stabilizing agents such as, e.g., HSA, the β-IFN of the invention is not aggregated (e.g., complexed) with, e.g., HSA.

Compositions of the invention (either in liquid or lyophilized form) also offer the advantage of being stable under ambient temperature storage conditions. Liquid formulations, therefore, do not need to refrigerate during storage and distribution. The invention provides a non-toxic, pharmaceutically acceptable solvent for IFN-β, particularly unglycosylated IFN-β, which provides a stable and soluble protein before, during and after lyophilization.

The term human “IFN-β” as used herein encompasses natural human IFN-β as well as recombinantly produced human IFN-β. Naturally occurring IFN-β includes that produced by fibroblast cells, e.g., human foreskin fibroblasts. Recombinant human IFN-β can be produced in any of a variety of host cells, either in a glycosylated form (e.g., in mammalian cells) or in an unglycosylated form (e.g., in bacterial cells). Typical host cells include, e.g., mammalian cells, in particular Chinese hamster ovary cells (see, e.g., U.S. Pat. No. 5,376,567). In a preferred embodiment, the IFN-β is produced in bacterial cells, preferably E. coli. Methods for producing heterologous proteins recombinantly are conventional and are described, e.g., in Sambrook, J. et al (1989). Molecular Cloning, a Laboratory Manual. Cold Spring Harbor Laboratory Press, Cold Spring Harbor, N.Y.; Ausubel, F. M. et al (1995). Current Protocols in Molecular Biology, N.Y., John Wiley & Sons; and Davis et al. (1986), Basic Methods in Molecular Biology, Elsevir Sciences Publishing, Inc., New York. See also also U.S. Pat. Nos. 5,004,605, 4,450,103, 4,315,852, 4,343,735 and 4,343,736. The invention also encompasses IFN-β analogs. A preferred IFN-β analog is the human recombinant cysteine-replaced mutein, IFN β-1b, which contains a serine residue in place of the natural unpaired cysteine residue at amino acid 17, as disclosed, e.g., in U.S. Pat. No. 4,588,585.

The amount of IFN-β in a liquid formulation that is to be stored as a liquid is preferably about 0.25 mg/mL to about 25.0 mg/ml, more preferably from about 0.5 mg/mL to about 10.0 mg/mL, and most preferably from about 1.0 mg/mL to about 10.0 mg/mL. Within the most preferred range of amounts, the most preferred amount in a liquid formulation that is to be stored as a liquid is about 5.0 mg/mL. The amount of IFN-β in a liquid formulation that is to be lyophilized for storage as a lyophilizate is preferably from about 0.25 mg/mL to about 25.0 mg/mL, more preferably from about 0.5 mg/mL to about 10.0 mg/mL, and most preferably from about 1.0 mg/mL to about 10.0 mg/mL. Within the most preferred range of amounts, the most preferred amount in a liquid formulation that is to lyophilized for storage as a lyophilizate is about 5.0 mg/mL.

“Biologically active” IFN-β or “biological activity” of IFN-β (or IFN-β analogs), as used herein, refers to determination of biological activity of IFN-β in a cytopathic effect (CPE)-inhibition assay. Such an assay measures the level of inhibition of viral cytopathic effect by interferon. CPE-inhibition assays are described in W. E. Stewart, The Interferon System, Springer-Verlag, New York, 1979. Specifically, the WISH-CPE assay system may be employed as described in S. E. Grossberg et al., “Biological and immunological assays of human interferons,” Manual of Clinical Immunology (1986), 3rd ed., N. R. Rose, H. Friedman and J. L. Fahley (eds), Washington, D.C., pp. 295-299. In addition, other activity detection systems, such as the MxA Induction Assay described in E. Pungor, Jr. et al., Journal of Interferon and Cytokine Research (1998), Vol. 18, pp. 1025-1030, and J. Files et al., Journal of Interferon and Cytokine Research (1998), Vol. 18, pp. 1019-1024, may be employed.

The biological activity of the IFN-β in the formulations of the invention as measured in a CPE-inhibition assay is preferably from about 0.75×10⁷ IU/mg to about 1.2×10⁸ IU/mg, more preferably from about 1.0×10⁷ IU/mg to about 4.5×10⁷ IU/mg, and most preferably at about 3.0×10⁷ IU/mg.

The concentration of glycine in a liquid formulation that is to be stored as a liquid or that is to be lyophilized for storage as a lyophilizate is preferably from about 1 milliMolar (mM) to about 100 mM, more preferably from about 5 mM to about 50 mM, and most preferably at about 20 mM.

For storage as a liquid formulation, it is contemplated that the IFN-β composition is sufficiently stable such that at least about 50%, preferably at least about 75%, and more preferably at least about 90% of the biological activity is retained after storage of the liquid formulation at 4° C. for at least 6 months, preferably at least 9 months, and more preferably at least one year. It is also contemplated that the IFN-β composition is sufficiently stable such that at least about 50%, preferably at least about 75%, and more preferably at least about 90% of the biological activity is retained after storage of the liquid formulation at ambient temperature (˜25° C.) for at least about 6 months, preferably for at least about 9 months, and more preferably for at least about 12 months.

For storage as a lyophilizate, it is contemplated that the IFN-β composition is sufficiently stable such that at least about 50%, preferably at least about 75%, and more preferably at least about 90% of the biological activity is recoverable in soluble form after storage of the lyophilizate at ambient temperature (approximately 25° C.) for at least 2 months, preferably at least 4 months, more preferably at least 6 months and most preferably at least 12 months. It is also contemplated that the IFN-β composition is sufficiently stable such that at least about 50%, preferably at least about 75%, and more preferably at least about 90% of the biological activity is recoverable in soluble form after storage of the lyophilizate at about 37° C. for at least about 6 months, preferably for at least about 9 months, and more preferably for at least about 12 months.

It is a further aspect of the invention that the IFN-β compositions of invention, as either the liquid formulation or as the lyophilizate, are substantially free of detergent and/or solubilizer, e.g., used in the isolation of the protein from the production system. The invention particularly relates to such compositions of recombinantly-produced, unglycosylated IFN-β that are substantially free of detergent and/or solubilizer used in the isolation of protein from the bacterial host. By “substantially free” is meant that such IFN-β compositions have associated with them a content of detergent and/or solubilizer of ≦50 ppm, preferably ≦25 ppm, more preferably ≦10 ppm, even more preferably ≦5 ppm, and most preferably ≦2 ppm. In a preferred embodiment, the amount of detergent is undetectable. For example, for SDS, the lowest amount of SDS which can be detected is about 25 ppm; therefore, an “SDS-free” composition is said to comprise ≦25 ppm of SDS. Compositions which are substantially free of detergent and/or solubilizer are sometimes referred to herein as being in the “substantial absence of” detergent and/or solubilizer or as having “substantially all” of the detergent and/or solubilizer removed from them.

The invention also relates to stable lyophilizates of IFN-β that may be reconstituted in water (e.g., WFI) or other pharmaceutically acceptable aqueous solutions in the absence of substantial amounts of SDS or other detergents/solubilizers such as, for example, Zwit 314, to yield substantially soluble and biologically active IFN-β. Particularly preferred are lyophilizates that may be reconstituted in parenterally administrable aqueous solutions. The solution for reconstitution of the lyophilizate may contain other pharmaceutically acceptable excipients as desired and as are well known in the art.

The invention also relates to formulations of IFN-β which are suitable for administration as an aerosol. These can be formulated from liquid preparations or from lyophilizates, either directly as a powder or after reconstitution with an appropriate liquid.

The glycine buffered composition can also contain additional conventional pharmaceutically acceptable excipients which provide, for example, improved handling properties. Bulking agents such as mannitol or sucrose, for example, can be in amounts which improve the lyophilization characteristics of the IFN-β/glycine buffered solution. The use of mannitol in combination with sucrose is also contemplated. The amount of mannitol employed is preferably less than about 50% (w/v), more preferably about 1.0% to about 5.0% (w/v), and most preferably about 2.0% (w/v). When mannitol is employed in combination with sucrose the ratio of manuitol/sucrose employed is preferably about 50 parts mannitol to 50 parts sucrose, more preferably about 75 parts mannitol to about 25 parts sucrose, most preferably about 100 parts mannitol to about 0 parts sucrose. The total amount of mannitol plus sucrose is preferably about 1.0% (w/v) to about 5.0% (w/v), more preferably about 2.0% (w/v).

In a preferred embodiment, IFN-β is bacterially-produced and is recovered from its bacterial host by a process which removes substantially all of the solubilizer, e.g., SDS, used in isolating the IFN-β from the bacterial inclusion bodies, and which yields a substantially biologically active IFN-β. Such methods are taught, e.g., in U.S. Pat. Nos. 4,462,940 and 5,643,566, and in particular in U.S. Pat. No. 5,004,605.

The compositions containing IFN-β dissolved in a glycine buffered solution, lyophilizates thereof, and lyophilizates reconstituted with water or other conventional pharmaceutically acceptable aqueous media are useful in the same manner as conventional pharmaceutical compositions containing IFN-β. For example, they can be administered to mammals, including humans, for the treatment of various diseases and conditions, e.g., viral diseases, cancer, multiple sclerosis, etc. Suitable amounts of IFN-β and regimens of administration, including routes and frequency of administration for treatment of various diseases and conditions, are well known in the art and can be routinely determined by the skilled practitioner. A dosage amount and schedule may be optimized for the individual patient. Optimization of dosage can be determined by monitoring clinical symptoms. Effective dosages are, for example, those which substantially alleviate the clinical symptoms, and/or slow the progression of, the disease.

The IFN-β preparation in accordance with the invention can be formulated in conventional ways standard in the art for administration of protein substances. Formulations of the invention are pharmaceutically acceptable for parenteral or non-parenteral delivery; are sterile; and/or are prepared and/or stored in a container (e.g., a vial, ampoule, syringe, etc.) which is suitable for administration to a patient (e.g., is injectable). One embodiment of the invention is a kit comprising: a) a container which contains a lyophilized preparation of IFN-β according to the invention, and b) a container which contains a suitable sterile aqueous solution for reconstitution of the lyophilizate, e.g., sterile water, which is preferably free of pyrogens of trace minerals. In a most preferred embodiment, the water is USP grade water for injection (WFI).

Administration by injection or inhalation with a pharmaceutically acceptable carrier or excipient, either alone or in combination with another agent, is preferred. Suitable formulations include solutions or suspensions, or emulsions or solid compositions for reconstitution into injectables or liquid aerosol formulations. Acceptable pharmaceutical carriers are those which dissolve the IFN-β or hold it in suspension and which are not toxic to the patient. Those skilled in the art will know, or be able to ascertain with no more than routine experimentation, particular suitable pharmaceutical carriers for this composition. See, e.g., U.S. Pat. Nos. 4,462,940, 5,643,566 and 5,004,605. Liquid aerosol formulations can be prepared according to the methods employed in, e.g., U.S. Pat. Nos. 5,941,240 and 5,558,085.

All materials for the expression, isolation and formulation of IFN-β and IFN-βₛₑᵣ₁₇ according to the invention are well known in the art. For example, the expression of human IFN-β in Escherichia coli is disclosed in Taniguchi et al., Proc. Natl. Acad. Sci. USA (1980), Vol. 77, pp. 5230-5233, and the expression of human IFN-β in Chinese hamster ovary cells is disclosed in U.S. Pat. No. 5,376,567. IFN-β analogs, such as the human recombinant cysteine-replaced mutein, IFN β-1b, which contains a serine residue in place of the natural unpaired cysteine residue at amino acid 17, are disclosed, e.g., in U.S. Pat. No. 4,588,585. Suitable purification and formulation methods (but not identical formulation ingredients) are disclosed in U.S. Pat. No. 4,462,940, U.S. Pat. No. 5,004,605, U.S. Pat. No. 5,702,669, and U.S. Pat. No. 5,643,566, which are all incorporated herein in full by reference.

E. coli K12/MM294-1 carrying plasmid pSY2501, which produces IFN β-1b, is deposited with the American Type Culture Collection, 12301 Parklawn Dr., Rockville, Md., 20852, U.S.A., under ATCC No. 39517.

### BRIEF DESCRIPTION OF THE FIGURES

FIG. 1 is a comparison of the RP-HPLC chromatograms of 0.6 mg/mL IFN β-1b in 100 mM glycine buffer, pH 3.0 at t=0 (Panel A) and at t=1 week, 37° C. (Panel B).

FIG. 2 is a graph of the lyophilization cycle for formulations of 0.1 mg/mL IFN β-1b in 100 mM glycine buffer, pH 3.0 containing either 4% mannitol (w/v) or 4% mannitol (w/v) and 1% sucrose (w/v).

FIG. 3 is a comparison of the RP-HPLC chromatograms of 0.1 mg/mL IFN β-1b in 100 mM glycine buffer, pH 3.0 with 4% mannitol, lyophilized and then reconstituted at the following time points: (1) prelyophilization; (2) reconstituted a t=0; (3) reconstituted at 25 weeks, 4° C.; (4) reconstituted at 25 weeks, 25° C.; (5) reconstituted at 25 weeks, 37° C.; (6) reconstituted at 2 weeks, 50° C.

FIG. 4 is a comparison of the RP-HPLC chromatograms of 0.1 mg/mL IFN β-1b in 100 mM glycine buffer, pH 3.0 with 4% mannitol, lyophilized and then reconstituted at the following time points: (1) reconstituted a t=0; (2) reconstituted at 8 weeks, 4° C.; (3) reconstituted at 25 weeks, 4° C.

FIG. 5 is a comparison of the RP-HPLC chromatograms of 0.1 mg/mL IFN β-1b in 100 mM glycine buffer, pH 3.0 with 4% mannitol, lyophilized and then reconstituted at the following time points: (1) reconstituted a t=0; (2) reconstituted at 8 weeks, 25° C.; (3) reconstituted at 25 weeks, 25° C.

FIG. 6 is a comparison of the RP-HPLC chromatograms of 0.1 mg/mL IFN β-1b in 100 mM glycine buffer, pH 3.0 with 4% mannitol, lyophilized and then reconstituted at the following time points: (1) reconstituted a t=0; (2) reconstituted at 8 weeks, 37° C.; (3) reconstituted at 25 weeks, 37° C.

FIG. 7 is a comparison of the RP-HPLC chromatograms of 0.1 mg/mL IFN β-1b in 100 mM glycine buffer, pH 3.0 with 4% mannitol and 1% sucrose, lyophilized and then reconstituted at the following time points: (1) reconstituted at t=0; (2) reconstituted at 8 weeks, 4° C.; (3) reconstituted at 25 weeks, 4° C.

FIG. 8 is a comparison of the RP-HPLC chromatograms of 0.1 mg/mL IFN β-1b in 100 mM glycine buffer, pH 3.0 with 4% mannitol and 1% sucrose, lyophilized and then reconstituted at the following time points: (1) reconstituted at t=0; (2) reconstituted at 8 weeks, 25° C.; (3) reconstituted at 25 weeks, 25° C.

FIG. 9 is a comparison of the RP-HPLC chromatograms of 0.1 mg/mL IFN β-1b in 100 mM glycine buffer, pH 3.0 with either 4% mannitol (Formulation 1) or 4% mannitol and 1% sucrose (Formulation 2), lyophilized and then reconstituted at t=0.

FIG. 10 is a comparison of the RP-HPLC chromatograms of 0.1 mg/mL IFN β-1b in 100 mM glycine buffer, pH 3.0 with either 4% mannitol (Formulation 1) or 4% mannitol and 1% sucrose (Formulation 2), lyophilized and then reconstituted at t=25 weeks, 4° C.

FIG. 11 is a comparison of the RP-HPLC chromatograms of 0.1 mg/mL IFN β-1b in 100 mM glycine buffer, pH 3.0 with either 4% mannitol (Formulation 1) or 4% mannitol and 1% sucrose (Formulation 2), lyophilized and then reconstituted at t=25 weeks, 25° C.

FIG. 12 is a comparison of the RP-HPLC chromatograms of 0.1 mg/mL IFN β-1b in 100 mM glycine buffer, pH 3.0 with either 4% mannitol (Formulation 1) or 4% mannitol and 1% sucrose (Formulation 2), lyophilized and then reconstituted at t=8 weeks, 37° C.

FIG. 13 is an image of an SDS-PAGE analysis of lyophilized and reconstituted reduced samples of 0.1 mg/mL IFN β-1b in 100 mM glycine buffer, pH 3.0 with either 4% mannitol (Formulation 1) or 4% mannitol and 1% sucrose (Formulation 2), after storage for 25 weeks at the indicated temperatures. The samples are run in duplicate. Lanes containing a prelyophilization sample, a t=0 sample, a molecular weight marker, and an IFN-β standard are also indicated.

FIG. 14 is an image of an SDS-PAGE analysis of lyophilized and reconstituted non-reduced samples of 0.1 mg/mL IFN β-1b in 100 mM glycine buffer, pH 3.0 with either 4% mannitol (Formulation 1) or 4% mannitol and 1% sucrose (Formulation 2), after storage for 25 weeks at the indicated temperatures. The samples are run in duplicate. Lanes containing a prelyophilization sample, a t=0 sample, a molecular weight marker, and an IFN-β standard are also indicated.

FIG. 15 is an image of an SDS-PAGE analysis of lyophilized and reconstituted samples of 0.1 mg/mL IFN β-1b in 100 mM glycine buffer, pH 3.0 with either 4% mannitol (Formulation 1) or 4% mannitol and 1% sucrose (Formulation 2), after storage at 50° C. for two weeks. Samples are reduced or non-reduced as indicated.

FIG. 16 is a graph of MxA induction results for test samples of 0.1 mg/mL IFN β-1b in 100 mM glycine buffer, pH 3.0 with either 4% mannitol (Formulation 1) or 4% mannitol and 1% sucrose (Formulation 2) lyophilized and then reconstituted after storage at the indicated temperature for the indicated time.

Without further elaboration, it is believed that one skilled in the art can, using the preceding description, utilize the present invention to its fullest extent. The following preferred specific embodiments are, therefore, to be construed as merely illustrative and not limitative of the remainder of the disclosure in any way whatsoever.

In the foregoing and in the following examples, all temperatures are set forth uncorrected in degrees Celsius; and, unless otherwise indicated, all parts and percentages are by weight.

In the foregoing and in the following examples, biological activity is expressed in International Units per milliliter of solution or IU/mL. An international unit is calculated as described in the Research Reference Reagent Note No. 35, published by the National Institute of Health, Bethesda, Md., in relation to the HulFN-β NIH reference reagent Gb 23-902-531 used as a standard.

### EXAMPLES

### Example 1

### Solubility and Stability as a Function of pH and Additives

A solution of purified IFN β-1b in 10 mM NaOH, pH 10.8, at a concentration of 0.3-0.5 mg/mL (9.6×10⁶-1.6×10⁷ IU/mL) is used as the starting material. The IFN β-1b is derived from E. coli fermentation of K12/MM294-1 carrying plasmid pSY2501 (ATCC 39517), purified according to the process described in U.S. Pat. No. 5,004,605. The pH of the starting IFN β-1b solution is adjusted instantaneously to the desired pH value by the addition of 1/10 volume of a 1 M stock solution of each additive which has previously been titrated to the desired pH value. The pH of the resulting IFN-β solutions is measured to ensure that no significant change in the pH of the additive solution occur as a result of dilution. Additional samples are prepared by adjusting the pH of IFN β-1b starting solutions to pH 5.0 or pH 6.5 with 1 N acetic acid in the presence or absence of 0.1% SDS. The samples are stored for 24 hours at 4° C., and the concentration of IFN β-1b remaining in solution is determined by an enzyme linked immunosorbent assay (ELISA) of the supernatant after centrifugation of the solution for 2 minutes at 12,000 rpm by methods similar to those described in P. N. Redlich et al., Proc. Natl. Acad. Sci. U.S.A. (1991), Vol. 88, pp. 404-4044; P. N. Redlich et al., The Journal of Immunology (1989), Vol. 143, No. 6, pp. 1887-1893; and P. N. Redlich et al., Eur. J. of Immunol. (1990). Vol. 20, pp. 1933-1939. The results of the ELISA analysis are presented in Table 1 below.

TABLE 1
Stability of IFN β-1b formulations (ELISA)

| | | % Recovery |
|---|---|---|
| | | after 24 hr |
| PH | Composition of buffer Formulation | at 4° C. |
| 10.8 | 10 mM NaOH | 78.0 |
| 10.0 | 100 mM glycine | 6.0 |
| 9.0 | 100 mM glycine | 3.0 |
| 8.0 | 100 mM sodium phosphate | 23.0 |
| 7.0 | 100 mM sodium phosphate | 10.0 |
| 7.0 | 100 mM sodium citrate | 11.0 |
| 6.5 | 10 mM sodium acetate, 0.1% SDS | 139.0 |
| 6.5 | 100 mM arginine | 11.0 |
| 5.0 | 10 mM sodium acetate, 0.1% SDS | 86.0 |
| 5.0 | 10 mM sodium acetate | 39.0 |
| 4.0 | 100 mM sodium acetate | 85.0 |
| 4.0 | 100 mM glycine | 50.0 |
| 4.0 | 100 mM sodium citrate | 4.0 |
| 4.0 | 100 mM aspartic acid | 88.0 |
| 3.0 | 100 mM glycine | 94.0 |

For samples with less than 30% recovery of IFN β-1b, a significant amount of visible precipitate forms immediately upon adjustment of the pH of the starting solution. The IFN β-1b is largely insoluble when adjusted to pH values below pH 10.8 and above pH 5.0 unless a solubilizing agent such as SDS is added. Solubility of the IFN-β can be maintained at pH 5.0 and below after 24 hours of storage at 4° C., depending on the additive with which the pH is adjusted. Both a sodium acetate buffer and an aspartic acid buffer at pH 4.0 solubilize and stabilize IFN-β significantly. However, a sodium citrate buffer does not maintain the solubility of IFN-β. Glycine buffered solution at pH 3.0 gives essentially complete recovery of IFN-β.

### Example 2

### Stability of IFN β-1b Solutions in 100 mM glycine buffer, pH 3.0.

Solutions of purified IFN β-1b in 100 mM glycine buffer, pH 3 (adjusted with hydrochloric acid) at concentrations of 0.6-1.1 mg/mL (1.9×10⁷-3.5×10⁷ IU/mL), derived from E. coli fermentation (as described in Example 1 above), are further evaluated for stability. Samples are stored at −70° C., 4° C. or 37° C. IFN β-1b stability is evaluated by reverse-phase high-pressure liquid chromatography (RP-HPLC) analysis, ELISA analysis, or WISH-CPE bioactivity analysis. The results are presented in Table 2.

TABLE 2
Stability of IFN-β in 100 mM glycine buffer, pH 3.0

| storage | | RP | ELISA | WISH |
|---|---|---|---|---|
| temp. | time | % of t = 0 | % of t = 0 | % of t = 0 |
| −70° C. | 3 days | 103 | 103 | 111 |
| 4° C. | 1 week | 120 | 120 | — |
| 4° C. | 2 week | 110 | 110 | — |
| 37° C. | 3 days | 97 | 97 | 197 |
| 37° C. | 1 week | 90 | 90 | 63 |

### Example 3

### Stability Studies on IFN-β Formulations

The following three formulations are evaluated for stability:
Formulation 1: 1.1 mg/mL IFN β-1b in 100 mM NaOAc buffer, pH 5.0.
Formulation 2:. 1.1 mg/mL IFN β-1b in 100 mM NaOAc buffer, pH 5.0+0.1% SDS.
Formulation 3:. 1.1 mg/mL IFN β-1b in 100 mM glycine buffer, pH 3.0.

The IFN β-1b is derived from E. coli fermentation as described in Example 1. Formulations 1-3 are prepared from G-25 pool of IFN β-1b by methods similar to those described in U.S. Pat. No. 4,462,940. All three formulations are filtered through a 0.2 μm filter attached to a syringe. A single filter is used for all formulations to minimize protein loss due to adsorption. The filter is rinsed with water between samples, and the SDS containing formulation is filtered last.

Stability of each formulation is evaluated after incubation in an osmotic pump (200 μL reservoir) for seven days at 37° C. Pumps are filled with approximately 215 μL of solution according to the pump directions. Pumps are weighed before and after filling to ensure complete filling. After seven days at 37° C., the pumps are transferred to a refrigerator and stored at 4° C. for six days before removal of the solutions from the pumps for analysis. Stability is also evaluated after storage of aliquots of each formulation in 0.5 mL Eppendorf tubes. The samples in Eppendorf tubes are incubated at 37° C. for 3 days and 7 days, and control samples are stored at 4° C. until the time of assay (approximately ten days later). The stability of each formulation to a freeze-thaw exposure is also evaluated. Four 100 μL aliquots of each formulation are removed from storage at each time point for analysis.

Stability is evaluated by RP-HPLC, ELISA, and WISH-CPE bioactivity. RP-HPLC and ELISA results are presented in Table 3, and WISH CPE bioassay results are presented in Table 4. RP-HPLC results for samples subjected to a freeze-thaw cycle (samples in Eppendorf tubes frozen at −70° C.) indicate that complete recovery IFN-β is obtained. RP-HPLC results for samples stored in Eppendorf tubes at 37° C. for one week show the following recoveries of IFN-β: 95% for formulation 1, 94% for formulation 2, and 86% for formulation 3. For samples stored in pumps at 37° C. for 1 week, RP-HPLC recovery results are as follows: 91% for formulation 1, 88% for formulation 2, and 71% for formulation 3. Recoveries after incubation in pumps are between 4-15% lower than in tubes under otherwise identical conditions. RP-FPLC chromatograms comparing formulation 4 at t=0 and at one week at 37° C. (in tubes or pumps) are shown in FIG. 1.

The data from the ELISA assay have a larger standard error than the data from the RP-HPLC assay. Nevertheless, the results obtained for formulations 1-3 are similar to those obtained by RP-HPLC analysis. Formulations 1 and 2 are stable for 1 week at 37° C., while formulation 3 loses approximately 25% of the initial activity.

The WISH-CPE bioassay has a large standard deviation. Additionally, samples are evaluated on different assay runs because of the limited number of samples that can be analyzed during one run. Formulations 1 and 2 appear to be stable to a freeze-thaw cycle and stable for 1 week at 37° C. in Eppendorf tubes or in pumps. For formulation 3, all results are significantly higher than expected and therefore, results for this formulation are difficult to evaluate.

Formulation 1 is also assayed for bioactivity by measuring down-regulation of TNF expression by activated monocytic cells in culture. These samples are assayed after incubation at 37° C. for 1 week and compared to samples stored at 4° C. This assay shows that formulation 1 loses 18% activity after storage at 37° C. for 1 week.

TABLE 3
RP-HPLC and ELISA Results

| | | | freeze- | | 7 days | 7 days |
|---|---|---|---|---|---|---|
| | | | thaw | 3 days at | at | at 37° C. |
| Prep # | Assay | t = 0 | −70° C. | 37° C. | 37° C. | (pump) |
| | RP-HPLC | μg/mL | μg/mL | μg/mL | μg/mL | μg/mL |
| 1 | | 1112 | 1224 | 1121 | 1063 | 1010 |
| 2 | | 1094 | 1107 | 1087 | 1031 | 960 |
| 3 | | 1076 | 1082 | 997 | 920 | 761 |
| | ELISA | | | | | |
| 1 | | 790 | 1620 | 1160 | 1010 | nt |
| | | | | (789) | (556) | |
| 2 | | (970) | 1100 | 1470 | 1530 | nt |
| | | | | (925) | (824) | |
| 3 | | 860 | 600 | 830 | 860 | nt |

Numbers in ( ) are from 2nd assay.
nt = not tested.

TABLE 4
WISH-CPE Bioactivity Results

| | | | | | 7 days |
|---|---|---|---|---|---|
| | | | | 7 days | at |
| | | freeze- | 3 days at | at | 37° C. |
| Prep | t = 0 | thaw | 37° C. | 37° C. | (pump) |
| # | IU/mL | IU/mL | IU/mL | IU/mL | IU/mL |
| 1 | 1.04 × 10⁸⁺ | 9.80 × 10⁷⁺ | 3.28 × 10⁸* | 1.36 × 10⁸* | 3.56 × |
| | | | | 3.14 × 10^{8#} | 10⁷* |
| 2 | 1.28 × 10⁸⁺ | 1.09 × 10⁸⁺ | 2.11 × 10⁸* | 2.05 × 10⁸* | 2.10 × |
| | | | | 4.69 × 10^{8#} | 10⁸* |
| 3 | 5.17 × 10⁷⁺ | 8.20 × 10^{8#} | 2.94 × 10⁸* | 3.10 × 10⁸* | 1.08 × |
| | | | | | 10⁹* |

*Samples were processed on the same day.
^{#}Samples were processed on the same day.
⁺Samples were processed on the same day.

### Example 4

### Stability of Lyophilized IFN β-1b in Glycine Buffer, pH 3.0

Lyophilized IFN-β is prepared from a solution of purified recombinant IFN β-1b in 100 mM glycine buffer, pH 3 (adjusted with hydrochloric acid) at a concentration of 0.1 mg/mL (3.2×10⁶ IU/mL). The IFN β-1b is derived from E. Coli fermentation as described in Example 1 above. Tubing vials (5.0 mL) are filled with 1 mL aliquots of the IFN β-1b solution. After the completion of lyophilization, while still under vacuum, gray butyl rubber stoppers are seated on the vials. Lyophilized IFN β-1b vials are stored at −70° C. or at 50° C. and reconstituted with 1 mL of water for injection at selected time points in the experiment. Reconstituted IFN β-1b samples are evaluated for IFN β-1b concentration by RP-HPLC analysis, ELISA analysis, or WISH CPE bioactivity analysis. The results of the evaluation of the samples of IFN β-1b concentration are presented in Table 5 below. Samples incubated at 50° C. are also analyzed by SDS-polyacrylamide gel electrophoresis (PAGE).

TABLE 5
Reconstituted IFN β-1b lyophilizate
(% of prelyophilization amount)

| storage temp. | time | RP-HPLC | WISH CPE | ELISA |
|---|---|---|---|---|
| −70° C. | 1 month | 87 | — | — |
| −70° C. | 6 month | 92 | 121 | — |
| 50° C. | 2 weeks | — | — | 85 |

The results demonstrate that lyophilized IFN β-1b formulated in a glycine buffered solution at pH 3.0 is stable for at least 6 months when stored at −70° C. and 2 weeks when stored at 50° C. Recoveries of ≧85% of IFN β-1b in reconstituted samples are measured at all time points. No change in the RP-HPLC profile of IFN β-1b is observed throughout the experiment and no degradation of IFN β-1b is detected by SDS-PAGE of lyophilized IFN β-1b in a glycine buffered solution at pH 3.0.

### Example 5

### Stability of Lyophilized IFN β-1b in Glycine Buffer+Mannitol or Glycine buffer+Mannitol/Sucrose

Lyophilized IFN β-1b formulations are prepared from solutions containing 0.1 mg/mL IFN β-1b, 100 mM glycine buffer, pH 3.0 and bulking agents consisting of either 4% mannitol (formulation 1) or 4% mannitol and 1% sucrose (formulation 2). The IFN β-1b is derived from E. coli fermentation as described in Example 1 above. Tubing vials (5.0 mL) are filled with 1 mL aliquots of the IFN β-1b solutions. After the completion of lyophilization while still under vacuum, gray butyl rubber stoppers are seated on the vials. Vials are stored at 4° C. and 25° C. and reconstituted with 1 mL of water for injection at selected time points throughout the experiment. Reconstituted samples are analyzed for IFN β-1b purity by reverse-phase HPLC (Table 6) and for bioactivity in a WISH CPE assay (Table 7).

TABLE 6
Reconstituted IFN β-1b lyophilizate as determined by RP-HPLC
Time of Reconstitution

| | Storage Temp. | | | | | |
|---|---|---|---|---|---|---|
| Formulation | (° C.) | 0 | 2 wks | 4 wks | 8 wks | 25 wks |
| 1 | 4 | 99 | 99 | 99 | 99 | 98 |
| 1 | 25 | 99 | 99 | 99 | 98 | 97 |
| 2 | 4 | 100 | 100 | 100 | 100 | 99 |
| 2 | 25 | 100 | 100 | 100 | 100 | 99 |

TABLE 7
Reconstituted IFN β-1b lyophilizate as determined
by WISH CPE ASSAY

| | Storage | | | |
|---|---|---|---|---|
| | Temp. | 0 | 8 wks | 25 wks |
| Formulation | (° C.) | IU/mL | IU/mL | IU/mL |
| 1 | 4 | 8.71 × 10⁶ | | 1.04 × 10⁷ |
| 1 | 25 | | 5.83 × 10⁶ | 1.75 × 10⁷ |
| 1 | 37 | | | 1.32 × 10⁷ |
| 2 | 4 | 3.82 × 10⁶ | | 4.92 × 10⁶ |
| 2 | 25 | | 6.30 × 10⁶ | 6.39 × 10⁶ |
| 2 | 37 | | | 5.40 × 10⁶ |

No significant change in IFN β-1b purity is detected for either formulation after storage at both 4°0 C. and 25° C. for up to 25 weeks. This result shows the unexpected superiority of the glycine buffered solution formulation in providing stable formulations for IFN β-1b, particularly lyophilized IFN β-1b, which is unglycosylated, in the absence of conventionally required stabilizing agents such as HSA.

### Example 6

### Further Studies on the Stability of Lyophilized IFN β-1b in Glycine Buffer+Mannitol or Glycine buffer+Mannitol/Sucrose

Two glycine based, non-HSA containing formulations of IFN β-1b are tested for stability after lyophilization. The two IFN β-1b formulations are as follows:
Formulation 1: 0.1 mg/mL IFN β-1b in 100 mM glycine buffer, pH 3.0, with 4% mannitol.
Formulation 2: 0.1 mg/mL IFN β-1b in 100 mM glycine buffer, pH 3.0, with 4% mannitol and 1% sucrose.

The IFN β-1b is derived from E. coli fermentation as described in Example 1. The IFN β-1b is prepared from a G-25 pool of IFN β-1b (prepared by methods described in Example 3 above) which is further purified over Q-Sepharose (G-25Q) to reduce the level of the carbohydrates. Approximately 75 vials of each formulation are filled for lyophilization and other vials of the IFN β-1b formulations are filled and stored at −70° C. for use as pre-lyophilization control samples. West Co. tubing vials (5 mL) are filled with 1.0 mL of formulated solution. Both formulations are lyophilized simultaneously and cycle data from the lyophilization is shown in FIG. 2. Samples are frozen to −43° C. and held for five hours. Primary drying is conducted at −35° C. for 25 hours, followed by −10° C. for four hours. Secondary drying is performed at 22° C. for 12 hours. Vials are stopped under full vacuum (˜50 mTorr) using non-siliconized 20 mM West 4416/50 stoppers.

Reconstitutions of IFN β-1b formulations are accomplished by the addition of 1.0 mL of water (WFI) to the vials. Vials of each formulation are stored at 4° C., 25° C., and 37° C., and samples are removed and reconstituted at t=2, 4, 8, 12 and 25 weeks to examine stability. Two vials of each formulation are reconstituted immediately post-lyophilization and frozen at −70° C. for later analysis at t=0 reconstitution controls. Reconstituted solutions are aliquoted into Eppendorf tubes (0.5 mL/tube) and stored at −70° C. until analysis.

Lyophilization of both formulations gives cakes with excellent appearance. No shrinkage is apparent and all cakes are white with a smooth top surface. Karl Fischer residual moisture is measured at only one time point, using vials which have been stored at −70° C. for approximately six months. Karl Fischer analysis is performed using an Aquastar colorimetric titrator, methanol as the extracting solvent, and non-pyridine containing reagents (Coulomat A and C, EM Science). The residual moisture results are similar for the two formulations: 0.63% for formulation 1 and 0.75% for formulation 2 (average of two vials for each formulation). Lyophilized samples of formulation 2 are observed to develop a yellow/brown color at higher incubation temperatures over time. Formulation 2 samples turn yellow between two and eight weeks storage at 37° C. Yellowing is not observed at the 25° C. and the 4° C. storage conditions for formulation 2. Formulation 1 samples remain white under all storage conditions. At all time points, samples of both formulations go into solution immediately (<30 seconds) upon reconstitution. The color of the resulting solutions is clear for all cakes, which are white. Formulation 2 samples which are yellow/brown give similarly colored solutions. No turbidity is observed for any samples.

Reconstituted samples are analyzed for stability by a variety of methods, including RP-HPLC, ELISA, SDS-PAGE, WISH CPE bioactivity, and MxA Induction Assay for bioactivity. RP-HPLC data are summarized in Tables 8 and 9 below. RP-HPLC data reported are the results of the average of values for two vials of each formulation. No significant differences are detected between any two duplicate vials. For each set of samples analyzed on a different date, pre-lyophilization and t=0 samples are analyzed on that date for comparison. Values for all pre-lyophilization and t=0 samples analyzed are averaged in the final tabulated data.

Chromatograms for formulation 1 are shown in FIGS. 3-6. The pre-lyophilization samples and the lyophilized samples reconstituted at t=0 give identical results. The chromatograms for samples stored at 4° C. for 25 weeks are essentially identical to the chromatograms for the pre-lyophilization samples and the t=0 samples, with the exception of a small new peak eluting at ˜6 minutes (FIGS. 3 and 4). For samples stored at 25° C. and 37° C., some broadening of the main IFN β-1b peak is observed, with a concomitant decrease in peak height, beginning at the 25 week and 8 week time points, respectively (FIGS. 5 and 6). In addition, an increase in material eluting after the main IFN β-1b peak (38-45 minutes) is observed. The small peak eluting at ˜6 minutes that is observed in the samples stored at 4° C. is also observed in samples stored at 25° C. and 37° C., and the peak is slightly larger in these samples (FIG. 3).

Chromatograms for formulation 2 are shown in FIGS. 7 and 8. The chromatograms for the prelyophilization samples and the lyophilized samples reconstituted at t=0 are identical. The chromatograms for samples stored at 4° C. for 25 weeks are essentially identical to the prelyophilization and t=0 sample chromatograms. For samples stored at 25° C., a very slight broadening of the main IFN β-1b is noted at the 25 week time point (FIG. 8).

Chromatograms comparing formulations 1 and 2 at the same conditions of time and temperature are shown in FIGS. 9-12. For samples of both formulations stored at 4° C. for 25 weeks, no significant changes in the chromatograms are detected when compared to the chromatograms of the pre-lyophilization and t=0 samples (FIGS. 3 and 10). For formulation 2 samples stored at 25° C., the extent of broadening of the main IFN β-1b peak at 25 weeks is slightly less than that observed for formulation 1 (FIG. 11). Also in contrast to formulation 1 samples, no increase in late eluting peaks is observed in the reverse phase profiles of formulation 2 samples stored at 25° C. For formulation 1 samples stored at 37° C., changes in the reverse phase profile are similar to but more extensive than changes observed in the profile of samples stored at 25° C. In contrast, formulation 2 samples stored at 37° C. show significantly increased degradation relative to samples stored at lower temperatures, for which essentially no degradation is detected (FIGS. 11 and 12).

In summary, the RP-HPLC analysis demonstrates that degradation of IFN β-1b in formulation 1 results in broadening of the main IFN β-1b peak and an increase in the amount of late-eluting peaks, which apparently correspond to late-eluting peaks that are present in the pre-lyophilization samples in low amounts. Therefore, the degradation path(s), as detected by RP-HPLC, appears to be similar over the examined temperature range, and the amount of degradation increases with increasing storage time and temperature. In contrast, there is a significantly increased degradation of formulation 2 at 37° C., relative to lower storage temperatures. Extensive degradation is detected as significant broadening of the main IFN β-1b peak. Unlike formulation 1, late-eluting material is not resolved as the peaks present at low levels in the prelyophilization samples. For formulation 2, the degradation pathways may be different for samples stored at elevated temperatures than for samples stored at lower temperatures.

Reconstituted samples at most storage time/temperature points are analyzed by ELISA and show highly variable results (Table 10). There appears to be a significant amount of ELISA activity remaining in samples of both formulations after storage for 25 weeks at any of the studied temperatures.

Reconstituted samples are also analyzed by SDS-PAGE. The analysis includes both reduced and non-reduced samples of formulations 1 and 2 that have been stored for 25 weeks at 4° C., 25° C. and 37° C., as well as prelyophilization and t=0 samples (FIGS. 13 and 14). Samples are analyzed on 10-20% Tricine gels (precast Novex). No new bands are detected for any samples compared to the prelyophilization controls. The only apparent change observed in IFN β-1b is the shift to slightly higher molecular weight of the IFN β-1b band for formulation 2 samples that have been stored at 37° C. for 25 weeks. This change is observed in both reduced and non-reduced samples. No change in IFN β-1b is detected for formulation 1 sample compared to the t=0 sample, while there is a slight decrease in mobility of the IFN β-1b band for the formulation 2 sample compared to the t=0 sample (FIG. 13). This change is IFN β-1b mobility appears to be similar to that observed for the formulation 2 sample stored at 37° C. for 25 weeks.

A limited number of samples are analyzed for bioactivity in both the WISH CPE bioassay and MxA Induction Assay (Table 11). These assays are used to determine whether or not bioactivity is retained under specific conditions. For the WISH CPE bioassay, the theoretical bioactivity of IFN β-1b at a concentration of ˜80 μg/mL (as indicated by RP-HPLC) is 3.8×10⁶ IU/mL. The results show good agreement with the expected activity for the pre-lyophilization and t=0 samples. The WISH CPE results indicate significant retention of bioactivity for both formulations at all storage temperatures. These same samples are also tested for bioactivity in the MxA Induction Assay (FIG. 16). Formulation 1 appears to retain significant bioactivity when stored at 4° C. and 37° C. The low value for the sample stored at 25° C. may also be due to a dilution error. Formulation 2 samples all show the same bioactivity as a percentage of the t=0 sample, suggesting no significant time dependent and/or temperature dependent loss of bioactivity.

TABLE 8
Reverse-phase HPLC Data for Formulation #1

| temp | time | μg/mL | % of t = 0 | % purity |
|---|---|---|---|---|
| 4° | prelyo | 88.0 | | 94.6 |
| | 0 | 83.4 | 100.0 | 94.0 |
| | 2 wks | 83.5 | 100.1 | 93.9 |
| | 4 wks | 82.5 | 98.9 | 93.7 |
| | 8 wks | 85.5 | 102.5 | 93.7 |
| | 25 wks | 82.0 | 98.3 | 93.3 |
| 25° | prelyo | 88.0 | | 94.6 |
| | 0 | 83.4 | 100.0 | 94.0 |
| | 2 wks | 82.5 | 98.9 | 93.6 |
| | 4 wks | 80.5 | 96.5 | 93.7 |
| | 8 wks | 81.5 | 97.7 | 93.1 |
| | 25 wks | 72.5 | 86.9 | 91.9 |
| 37° | prelyo | 88.0 | | 94.6 |
| | 0 | 83.4 | 100.0 | 94.0 |
| | 2 wks | 81.0 | 97.1 | 92.1 |
| | 4 wks | 77.0 | 92.3 | 91.2 |
| | 8 wks | 74.0 | 88.7 | 90.1 |
| | 25 wks | 64.5 | 77.3 | 91.2 |

TABLE 9
Reverse-phase HPLC Data for Formulation #2

| temp | time | ug/mL | % of t = 0 | % purity |
|---|---|---|---|---|
| 4° | prelyo | 87.0 | | 94.0 |
| | 0 | 85.2 | 100.0 | 94.0 |
| | 2 wks | 85.5 | 100.4 | 93.9 |
| | 4 wks | 87.5 | 102.7 | 93.9 |
| | 8 wks | 87.5 | 102.7 | 94.1 |
| | 25 wks | 81.0 | 95.1 | 93.4 |
| 25° | prelyo | 87.0 | | 94.0 |
| | 0 | 85.2 | 100.0 | 94.0 |
| | 2 wks | 89.0 | 104.5 | 93.9 |
| | 4 wks | 90.5 | 106.2 | 94.2 |
| | 8 wks | 91.5 | 107.4 | 94.2 |
| | 25 wks | 75.5 | 88.6 | 93.4 |
| 37° | prelyo | 87.0 | | 94.0 |
| | 0 | 85.2 | 100.0 | 94.0 |
| | 2 wks | 84.5 | 99.2 | 94.1 |
| | 4 wks | 81.0 | 95.1 | 93.9 |
| | 8 wks | 71.0 | 83.3 | 93.1 |
| | 25 wks | 63.5 | 74.5 | 66.8 |

TABLE 10
ELISA Results for Reconstituted Samples

| | | |
|---|---|---|
| Temp | Time | % of t = 0 |

Formulation #1

| | | |
|---|---|---|
| 4° C. | 4 wks | 56 |
| | 8 wks | 85 |
| | 12 wks | 68 |
| | 25 wks | 104 |
| 25° C. | 4 wks | 55 |
| | 8 wks | 215 |
| | 12 wks | 66 |
| | 25 wks | 105 |
| 37° C. | 4 wks | 79 |
| | 8 wks | 271 |
| | 12 wks | 49 |
| | 25 wks | 80 |

Formulation #2

| | | |
|---|---|---|
| 4° C. | 4 wks | 56 |
| | 8 wks | 276 |
| | 12 wks | 58 |
| | 25 wks | 97 |
| 25° C. | 4 wks | 61 |
| | 8 wks | 290 |
| | 12 wks | 50 |
| | 25 wks | 63 |
| 37° C. | 4 wks | 51 |
| | 8 wks | 246 |
| | 12 wks | 33 |
| | 25 wks | 49 |

TABLE 11
Bioactivity Results for Reconstituted Samples

| | | | |
|---|---|---|---|
| | | WISH CPE | MxA |
| temp | Time | IU/mL | % of t = 0 |

Formulation #1

| | | | |
|---|---|---|---|
| | prelyo | 6.55 × 10⁶ | |
| | 0 | 8.71 × 10⁶ | 100 |
| 4° C. | 25 wks | 1.04 × 10⁷ | 107 |
| 25° C. | 8 wks | 5.83 × 10⁶ | 87 |
| 25° C. | 25 wks | 1.75 × 10⁷ | 11 |
| 37° C. | 25 wks | 1.32 × 10⁷ | 117 |

Formulation #2

| | | | |
|---|---|---|---|
| | prelyo | 3.53 × 10⁶ | |
| | 0 | 3.82 × 10⁶ | 100 |
| 4° C. | 25 wks | 4.92 × 10⁶ | 68 |
| 25° C. | 8 wks | 6.30 × 10⁶ | 74 |
| 25° C. | 25 wks | 6.39 × 10⁶ | 73 |
| 37° C. | 25 wks | 5.40 × 10⁶ | 75 |

The preceding examples can be repeated with similar success by substituting the generically or specifically described reactants and/or operating conditions of this invention for those used in the preceding examples.

The entire disclosures of all applications, patents and publications, cited above and below, if any, are hereby incorporated by reference.

From the foregoing description, one skilled in the art can easily ascertain the essential characteristics of this invention, and without departing from the spirit and scope thereof, can make various changes and modifications of the invention to adapt it to various usages and conditions.

1. An pharmaceutical composition consisting essentially of biologically active IFN-β1b and of glycine buffer that achieves a pH of about 2 to about 4.

2. A composition according to claim 1, wherein the buffer achieves a pH of 3.0 to 3.5.

3. A composition according to claim 1, wherein the buffer achieves a pH of 2.8 to 3.2.

4. A composition according to claim 1, wherein the buffer achieves a pH of 2.9 to 3.1.

5. A composition according to claim 1, wherein the buffer achieves a pH of about 3.0.

6. A composition according to claim 1 further containing water.

7. A composition according to claim 1, wherein the buffer contains HCl.

8. A composition according to claim 1 further containing a pharmaceutically acceptable carrier.

9. A composition according to claim 1 that is sterile.

10. A composition according to claim 1, wherein 75% of the biological activity of the IFN-β-1b is retained after storage of the composition at 4° C. for at least 9 months.

11. A composition according to claim 1, wherein the IFN-β-1b is unglycosylated and is produced in a bacterial host.

12. A composition according to claim 1, wherein 75% of the biological activity of the IFN-β-1b is retained after storage of the composition at 37° C. for at least 9 months.

13. A composition according to claim 1, wherein the composition is substantially free of human serum albumin.

14. A composition according to claim 1, wherein the composition does not contain a detectable amount of a detergent.

15. A composition according to claim 1, wherein the concentration of biologically active IFN-β-1b is about 0.25 mg/ml to about 25 mg/ml.

16. A composition according to claim 1, wherein the concentration of biologically active IFN-β-1b is about 5 mg/ml.

17. A composition comprising about 5 mg/ml biologically active IFN β-1b in glycine buffer at about pH 3.0.

18. A composition according to claim 1, wherein the glycine buffer contains HCl.

19. A composition according to claim 1, wherein the IFN-β-1b is not in a form of a non-covalently associated aggregate.

20. A composition according to claim 1, wherein the glycine buffer is 100 mM glycine buffer.

21. A composition according to claim 1, wherein the glycine component is in a stabilizing effective amount.

22. A composition according to claim 1, wherein the glycine component is at a concentration of about 1 mM to about 100 mM.

23. A composition according to claim 1, wherein the glycine component is at a concentration of about 2-5 mM.

24. A composition according to claim 1 that is in a container for parenteral or subcutaneous administration.

25. A composition according to claim 1, wherein the parenteral or subcutaneous administration is by injection or inhalation.

26. A composition according to claim 1 that is lyophilized.

27. A composition according to claim 26, prepared by lyophilizing a composition consisting essentially of biologically active IFN-β-1b and of glycine buffer that achieves a pH of about 2 to about 4.

28. A kit comprising
a) a container which contains a lyophilized IFN-β-1b composition of claim 26 and
b) a container which contains sterile pyrogen-free water.

29. A kit according to claim 28, further containing in container of a) and/or b) and/or in a separate container, a pharmaceutically acceptable excipient.

30. A method of preparing a composition according to claim 1, comprising preparing a glycine buffer that achieves a pH of about 2-4 and bringing said buffer and IFN-β into a composition.

31. A method of preparing a composition according to claim 26, comprising lyophilizing a composition consisting essentially of biologically active IFN-β-1b and of glycine buffer that achieves a pH of about 2 to about 4.

32. A method of preparing a kit according to claim 27, comprising placing the lyophilized IFN-β-1b composition into a container.

33. A method of treating multiple sclerosis, comprising administering an effective amount of a composition according to claim 1 to a patient in need thereof.

34. A method of administering a composition according to claim 1 by parenterally or subcutaneously administering said composition to a patient in need thereof.

35. A method according to claim 34, wherein the administration is by injection or inhalation.
